(19) United States
(12) Patent Application Publication
(10) Pub. No.: US 2005/0273093 A1
(43) Pub. Date: Dec. 8, 2005

(54) Method of treating herniated intervertebral discs using cooled ablation

(75) Inventors: Mukund R. Patel, San Jose, CA (US); Gopal K. Chopra, San Francisco, CA (US)

Correspondence Address: Bingham McCuthen, LLP, Suite 1800, Three Embarcadero, San Francisco, CA, 94111-4067, US

(73) Assignee: SciMed Life Systems, Inc.

(21) Appl. No.: 10/861,164

(22) Filed: Jun. 4, 2004

(51) Int. Cl. A61B 18/14

(52) U.S. Cl. 606/41

(57) ABSTRACT

A method of treating an intervertebral disc is provided. The method comprises introducing a probe with an electrode into contact with the nucleus pulposus of the disc, and conveying radio frequency ablation energy from the electrode into the nucleus pulposus of the disc. For example, if the disc has a herniated region, the electrode can be placed into contact with the nucleus pulposus within the herniated region (e.g., by steering the electrode), and the ablation energy can be conveyed directly into the herniated region. The electrode can also be placed into contact near the center region of the disc, in which case, the ablation energy can be conveyed into the center region. In any event, tissue is removed, which may decompress the disc or provide some other therapeutic result. The method further comprises circulating a cooling medium through the probe in thermal contact with the electrode. By cooling the electrode, tissue is more efficiently ablated from the disc.

### FIELD OF THE INVENTION

The field of the invention pertains to medical devices and methods for treating intervertebral disc hernias, and more particularly, to decompressing herniated intervertebral discs using radio frequency (RF) ablation.

### BACKGROUND OF THE INVENTION

The spinal column consists of thirty-three bones called vertebra, the first twenty-four vertebrae of which make up the cervical, thoracic, and lumbar regions of the spine and are separated from each other by “pads” of tough cartilage called “intervertebral discs,” which act as shock absorbers that provide flexibility, stability, and pain-free movement of the spine.

FIGS. 1 and 2 illustrate a portion of a healthy and normal spine, and specifically, two vertebra 10 and two intervertebral discs 12 (only one shown). The posterior of the vertebra 10 includes right and left transverse processes 14R, 14L, right and left superior articular processes 16R, 16L, and a spinous process 18. Muscles and ligaments that move and stabilize the vertebra 10 are connected to these structures. The vertebra 10 further includes a centrally located lamina 20 with right and left lamina 20R, 20L, that lie inbetween the spinous process 18 and the superior articular processes 16R, 16L. Right and left pedicles 22R, 22L are positioned anterior to the right and left transverse processes 14R, 14L, respectively. A vertebral arch 24 extends between the pedicles 22 and through the lamina 20. The anterior of the vertebra 10 includes a vertebral body 26, which joins the vertebral arch 24 at the pedicles 22. The vertebral body 26 includes an interior volume of reticulated, cancellous bone (not shown) enclosed by a compact cortical bone 30 around the exterior. The vertebral arch 24 and vertebral body 26 make up the spinal canal (i.e., the vertebral foramen 32), which is the opening through which the spinal cord 34 and epidural veins (not shown) pass. Nerve roots 36 laterally pass from the spinal cord 34 out through the neural foramen 38 at the side of the spinal canal formed between the pedicles 22. Structurally, the intervertebral disc 12 consists of two parts: an inner gel-like nucleus (nucleus pulposus) 40 located centrally within the disc 12, and tough fibrous outer annulus (annulus fibrosis) 42 surrounding the nucleus 40.

A person may develop any one of a variety of debilitating spinal conditions and diseases. One of the more common spinal conditions results when an intervertebral disc become herniated, as illustrated in FIG. 3. A herniation of an intervertebral disc may occur suddenly or gradually over a period of time, as illustrated in FIGS. 4-7. First, the outer wall of the pre-herniated disc 12′ (i.e., the annulus fibrosis 42) becomes weakened due to the chemical changes associated with aging. As a result, the disc 12′ degenerates and begins to bulge out in the posterior direction, as illustrated in FIG. 4. It should be noted that, in some cases, such as trauma, degeneration of an intervertebral disc is not required for it to herniate. In either case, the annulus fibrosis 42 may eventually tear (as a result of sneezing, bending, or just through natural attrition), thereby allowing the soft inner part of the disc 12 (i.e., the nucleus pulposus 40) to bulge out, forming a prolapsed herniated region 48, as illustrated in FIG. 5. At this point, the nucleus pulposus 40 is completely contained by the annulus fibrosis 42. Eventually, as illustrated in FIG. 6, the nucleus pulposus 40 may extrude through the tear in the annulus fibrosis 42, resulting in an extruded herniated region 50. In some cases, as illustrated in FIG. 7, a portion of the nucleus pulposus becomes separated from the parent nucleus pulposus 40, resulting in a sequestered herniated region 52.

Whether the herniation is contained (in the case of a prolapsed herniation) or not contained (in the case of an extruded or sequestered herniation), the herniated region of the disc often pinches or compresses the adjacent dorsal root 36 against a portion of the vertebra 10, as illustrated in FIG. 3, resulting in weakness, tingling, numbness, or pain in the back, legs or arm areas. In addition to nerve root compression, any of the nucleus pulposus 40 that escapes the annulus fibrosis 42 may chemically irritate neural tissue.

Often, inflammation from disc herniation can be treated successfully by nonsurgical means, such as bed rest, therapeutic exercise, oral anti-inflammatory medications or epidural injection of corticosterioids, and anesthetics. In some cases, however, the disc tissue is irreparably damaged, in which case, surgery is the best option.

Discectomy, which involves removing all, or a portion, of the affected disc, is the most common surgical treatment for ruptured or herniated discs of the lumbar spine. In most cases, a laminotomy or laminectomy is performed to visualize and access the affected disc. Once the vertebrae, disc, and other surrounding structures can be visualized, the surgeon will remove the section of the disc that is protruding from the disc wall and any other offending disc fragments that may have been expelled from the disc. In some cases, the entire disc may be removed, with or without a bony fusion or arthroplasty (disc nucleus replacement or total disc replacement).

Open discectomy is usually performed under general anesthesia and typically requires at least a one-day hospital stay. During this procedure, a two to three-inch incision in the skin over the affected area of the spine is made. Muscle tissue may be separated from the bone above and below the affected disc, while retractors hold the wound open so that the surgeon has a clear view of the vertebrae and disc and related structures. The disc or a portion thereof, can then be removed using standard medical equipment, such as rongeurs and curettes.

Because open discectomy requires larger incisions, muscle stripping or splitting, more anesthesia, and more operating, hospitalization, and a longer patient recovery time, the trend in spine surgery is moving towards minimally invasive surgical techniques, such as microdiscectomy and percutaneous discectomy.

Microdiscectomy uses a microscope or magnifying instrument to view the disc. The magnified view may make it possible for the surgeon to remove herniated disc material through a smaller incision (about twice as small as that required by open discectomy) with smaller instruments, potentially reducing damage to tissue that is intended to be preserved.

Percutaneous discectomy is often an outpatient procedure that may be carried out by utilizing hollow needles or cannulae through which special instruments can be deployed into the vertebra and disc in order to cut, remove, irrigate, and aspirate tissue. X-ray pictures and a video screen and computer-aided workstation may be used to guide by the surgeon into the treatment region. Improved imaging and video or computer guidance systems have the potential to reduce the amount of tissue removal required to access and treat the injured tissue or structures. Sometimes an endoscope is inserted to view the intradiscal and perivertebral area.

As shown in FIG. 8, percutaneous access to the herniated disc 12′ may be provided by introducing a needle 60 (typically a 17 or 18 gauge needle) into the patient's spine. As is typical with most hernias, the herniated region 46 of the disc 12′ resides on the posterior side of the disc 12′. Due to the presence of the spinal cord 34 and the posterior portion of the vertebra, namely the lamina 20, introduction of the needle 60 from a direct posterior position cannot be easily accomplished. Thus, unlike open discectomy or microdiscectomy, which typically involves performing a laminectomy or laminotomy to directly access the herniated region of the disc, the herniated region 46 of the disc 12′ cannot be directly accessed using the percutaneous approach. As such, the needle 60 must be introduced into the disc 12′ via a standard extra-pedicular posterior-lateral approach.

A probe 62 with an ablative element 64, which may remove tissue using chemical, mechanical, or thermal/heat (radio frequency energy or laser) means, may then be introduced through the needle 60 and into the disc 12′ to remove nuclear tissue 40 from the center of the disc 12′, as illustrated in FIG. 9. This can typically be accomplished by moving the probe 62 back and forth during the ablation process to create channels 66 (where tissue has been removed) within the disc 12′. As a result, the disc 12′ decompresses, thereby relieving pressure between the herniated region 46 of the disc 12′ and the spinal cord 34 and nerve root 36. The extruded disc may not be effectively treated by decompressing the center of the disc. Also, sequestered hernias cannot be treated in this manner, since nuclear fragments will still remain in the spinal canal after decompression of the disc 12′.

Because the nuclear tissue 40 that is removed from the disc 12′ is relatively far away from the herniated disc portion, the effectiveness of the decompression may be limited. In the case of ablative means that uses RF energy, the amount of tissue ablated is limited by heat dispersion, and must be compensated for by moving the probe 64 within the disc 12′. Increasing generator output has been unsuccessful for increasing lesion diameter, because an increased wattage is associated with a local increase of temperature to more than 100° C., which induces tissue vaporization and charring. This, then, increases local tissue impedance, limiting RF deposition, and therefore heat diffusion and associated coagulation necrosis. The increased temperature may also have risk of nerve injury.

There, thus, remains a need to provide an improved means for percutaneously treating herniated intervertebral discs using RF tissue ablation.

### SUMMARY OF THE INVENTION

A method of treating an intervertebral disc is provided. The method comprises introducing a probe with an electrode into contact with the nucleus pulposus of the disc. In one method, the probe may be percutaneously introduced into the disc, but may alternatively be introduced into the disc in any one of a variety of other manners. The method further comprises conveying radio frequency ablation energy from the electrode into the nucleus pulposus of the disc. For example, if the disc has a herniated region, the electrode can be placed into contact with the nucleus pulposus within the herniated region (e.g., by steering the electrode), and the ablation energy can be conveyed directly into the herniated region. The electrode can also be placed into contact near the center region of the disc, in which case, the ablation energy can be conveyed into the center region. In any event, tissue is removed, which may decompress the disc or provide some other therapeutic result. The method further comprises circulating a cooling medium through the probe in thermal contact with the electrode. The cooling fluid may be chilled, e.g., to a temperature of 5° C.-10° C., or may be at room temperature. In one method, the cooling medium is circulated into thermal contact with the electrode during the ablation process. By cooling the electrode, tissue is more efficiently ablated from the disc.

In accordance with a second aspect of the present inventions, a method of treating an intervertebral disc having a herniated region is provided. The herniated region may be contained or not contained. The method comprises introducing a probe into contact with the disc. In one method, the probe may be percutaneously introduced into the disc, but may alternatively be introduced into the disc in any one of a variety of other manners. The probe has a steerable distal tip and an ablative element located on the distal tip. The method further comprises steering the distal tip of the probe into contact with the nucleus pulposus in the herniated region (e.g., by operating a steering mechanism on the probe), and conveying ablation energy from the ablative element directly into the herniated region. The ablation energy may be RF ablation energy, or some other type of ablation energy, such as laser or mechanical energy. A cooling medium can optionally be circulated through the probe in thermal contact with the ablative element.

Other objects and features of the present invention will become apparent from consideration of the following description taken in conjunction with the accompanying drawings.

### BRIEF DESCRIPTION OF THE DRAWINGS

The drawings illustrate the design and utility of preferred embodiments of the present invention. It should be noted that the figures are not drawn to scale and that elements of similar structures or functions are represented by like reference numerals throughout the figures. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a perspective view of a portion of a spine;

FIG. 2 is a top view of a vertebra with a healthy intervertebral disc;

FIG. 3 is a top view of a vertebra with a herniated intervertebral disc;

FIG. 4 is a top view of a degenerated intervertebral disc;

FIG. 5 is a top view of an intervertebral disc with a prolapsed hernia;

FIG. 6 is a top view of an intervertebral disc with an extruded hernia;

FIG. 7 is a top view of an intervertebral disc with a sequestered hernia;
prior art tissue removal probe;

FIG. 8 is top view illustrating the percutaneous introduction of a needle into a herniated intervertebral disc;

FIG. 9 is a top view illustrating the introduction of an ablation probe into the disc of FIG. 8 and subsequent treatment of the disc with the ablation probe; another prior art tissue removal probe;

FIG. 10 is a plan view of a tissue ablation system arranged in accordance with a preferred embodiment of the present invention;

FIG. 11 is a partially cutaway side view of an ablation probe used in the system of FIG. 10;

FIG. 12 is a cross-sectional view of the ablation probe of FIG. 11, taken along the line 12-12;

FIG. 13 is a cross-sectional view of the ablation probe of FIG. 11, taken along the line 13-13; and

FIGS. 14A-14E are top views showing a method of using the tissue removal system of FIG. 10 to treat a herniated intervertebral disc.

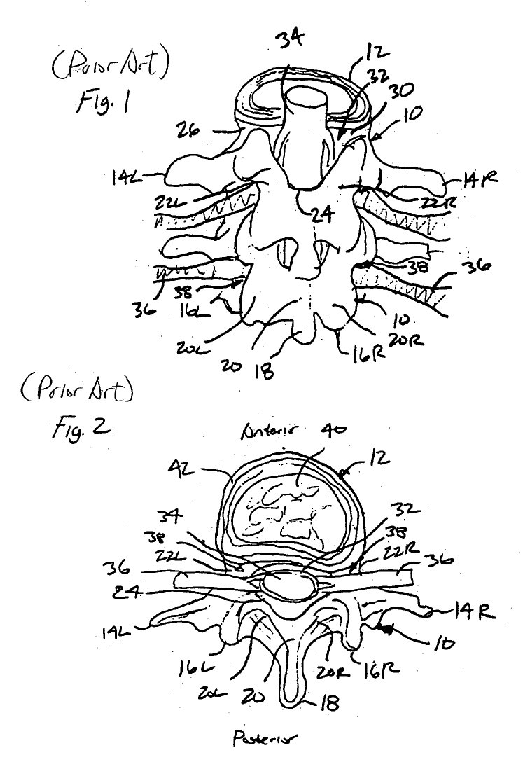

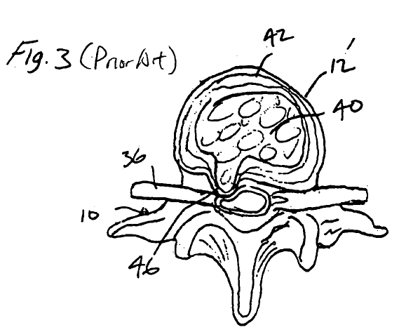

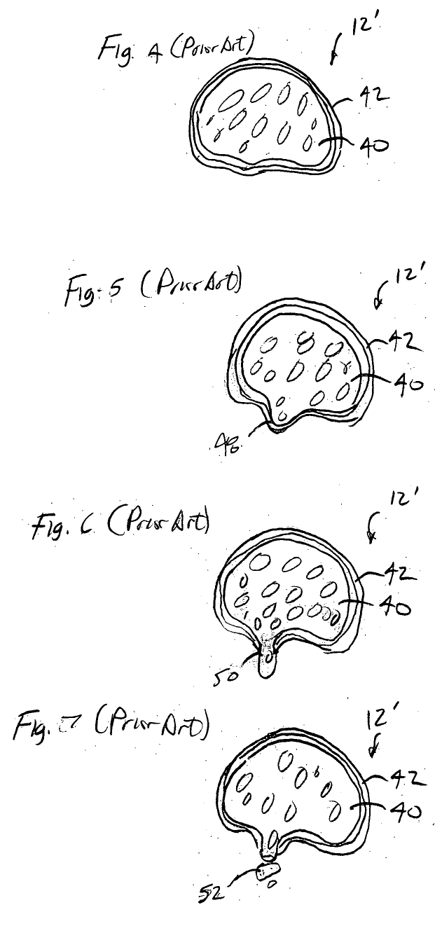

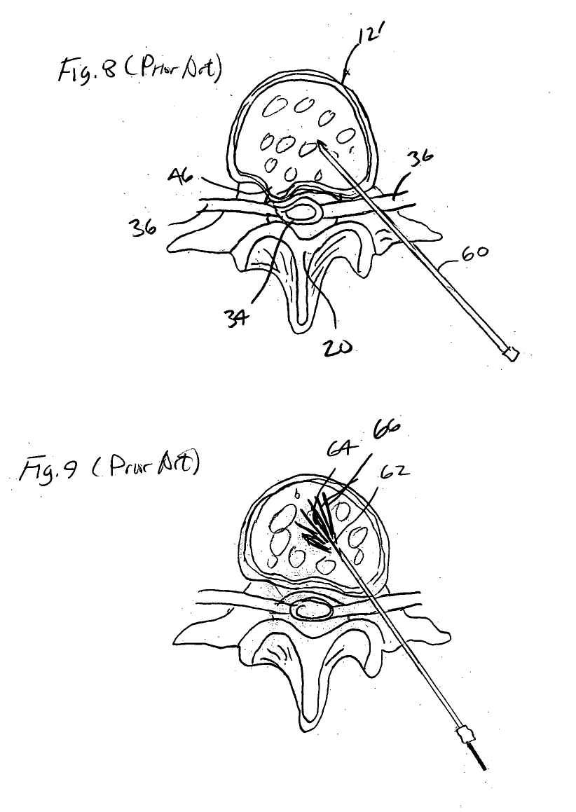

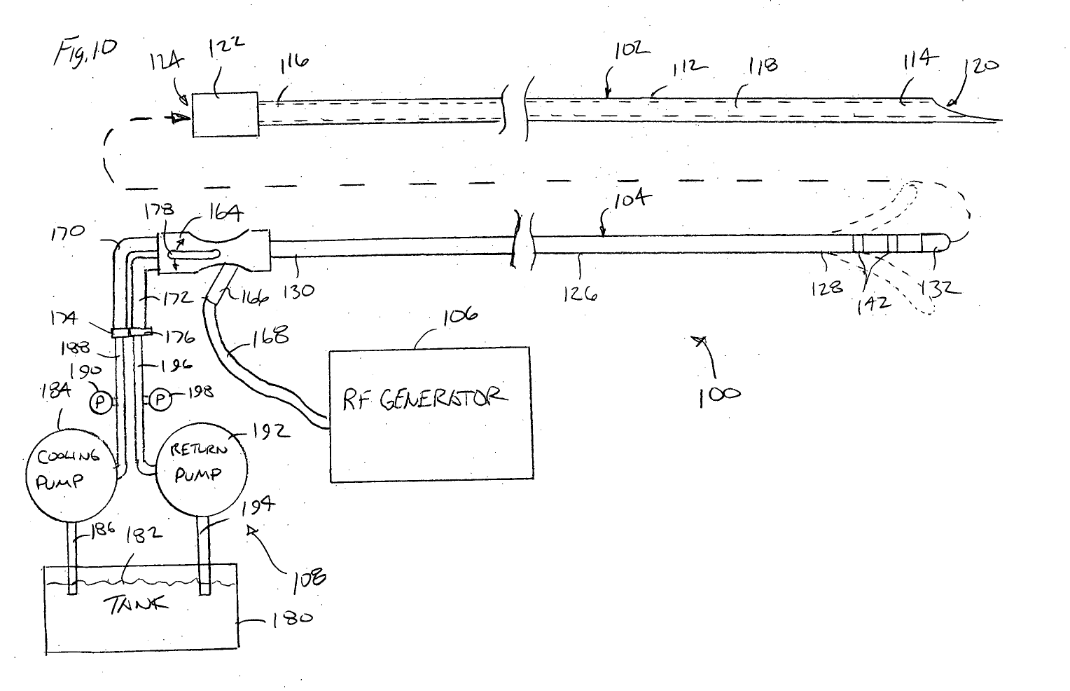

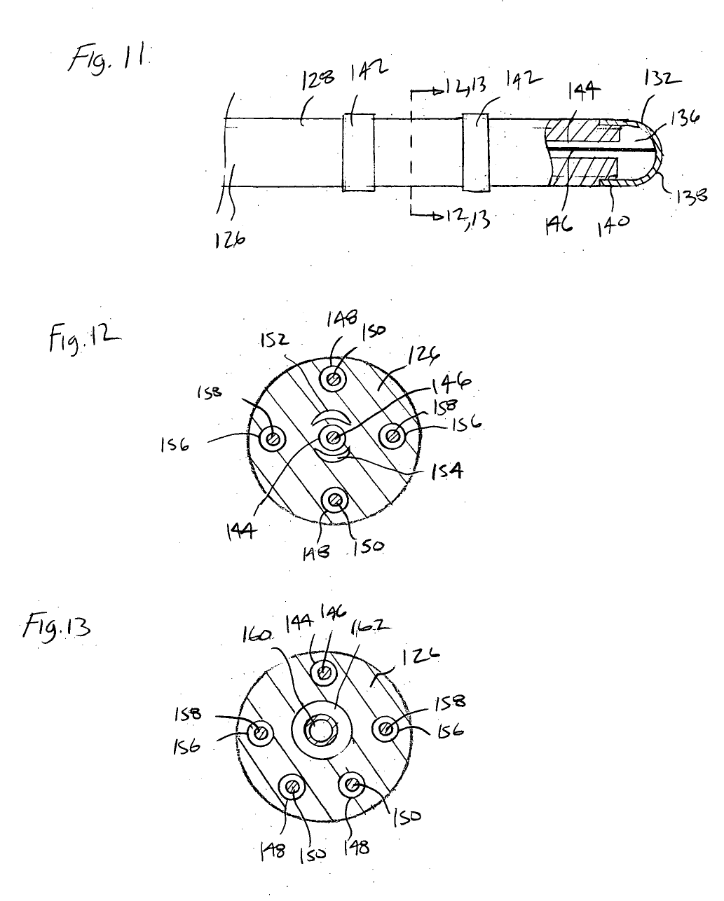

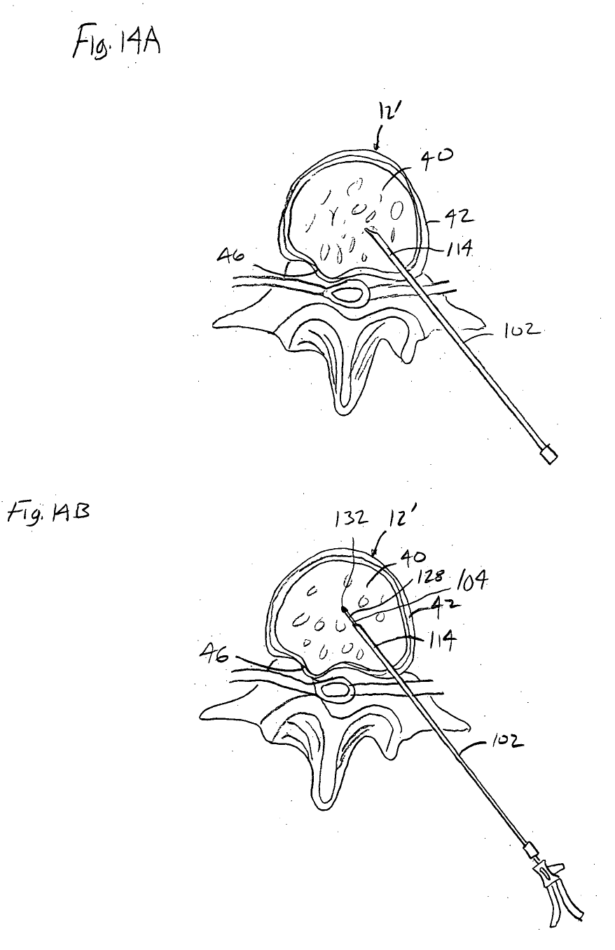

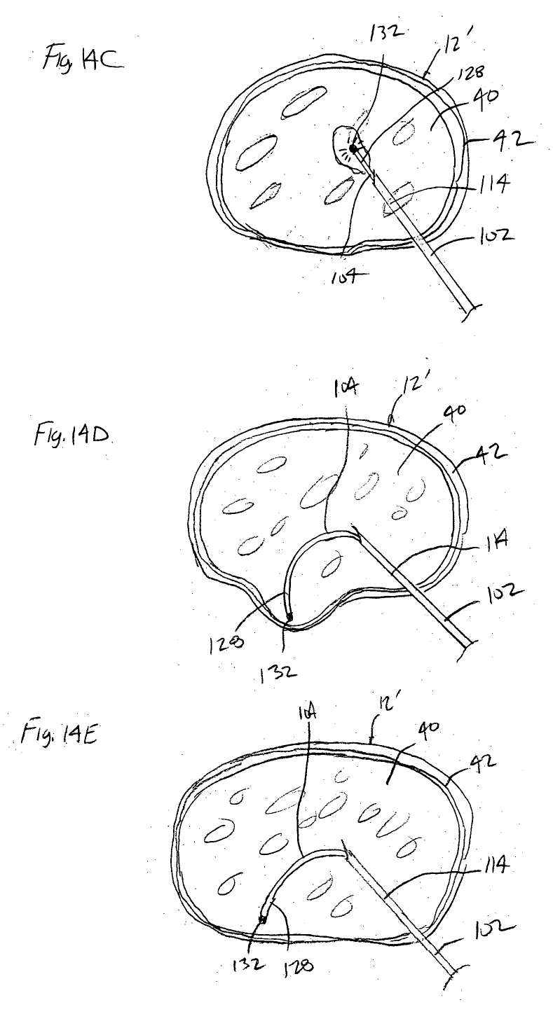

### DETAILED DESCRIPTION OF THE EMBODIMENTS

FIG. 10 illustrates a cooled tissue ablation system 100 constructed in accordance with a preferred embodiment of the present inventions. The system 100 generally comprises an introducer cannula 102 for providing percutaneous access to an intervertebral disc, a tissue ablation probe 104 for ablating selected tissue within the intervertebral disc, a radio frequency (RF) generator 106 configured to deliver RF ablation energy to the probe 104, and a pump assembly 108 configured to deliver a cooling medium to the probe 104 during the ablation process.

The cannula 102 comprises a shaft 112 having a distal end 114 and proximal end 116, a lumen 118 (shown in phantom) terminating in an exit port 120 at the distal end 114 of the cannula shaft 112, and a handle 122 mounted on the proximal end 116 of the cannula shaft 112. The handle 122 defines an entry port 124 in communication with the cannula lumen 118. To facilitate introduction through tissue, the cannula shaft 112 is preferably stiff (e.g., it can be composed of a stiff material, or reinforced with a coating or a coil to control the amount of flexing), so that the cannula shaft 112 can penetrate the tissue without being damaged. The materials used in constructing the cannula shaft 112 may comprise any of a wide variety of biocompatible materials. In a preferred embodiment, a radiopaque material, such as metal (e.g., stainless steel, titanium alloys, or cobalt alloys) or a polymer (e.g., ultra high molecular weight polyethylene) may be used, as is well known in the art. Alternatively, if supported by a rigid member during introduction into the tissue, the cannula shaft 112 may be flexible. The handle 122 is preferably composed of a durable and rigid material, such as medical grade plastic, and is ergonomically molded to allow a physician to more easily manipulate the cannula 102.

The outer diameter of the cannula shaft 112 is preferably less than ½ inch, but other dimensions for the outer diameter of the cannula shaft 112 may also be appropriate. The cannula lumen 118 should have an inner diameter so as to allow the tissue ablation probe 104 to be slidably housed therein, as will be described in further detail below. In the illustrated embodiment, the profile of the cannula lumen 118 is circular, but can be other shapes as well. In the illustrated embodiment, the distal tip of the cannula shaft 112 tapered or sharpened to facilitate its introduction through tissue. Alternatively, the distal tip of the cannula shaft 112 is blunt, in which case, a stylet (not shown) can be introduced through the cannula lumen 118 to provide an independent means for boring through tissue. In this manner, tissue cores will not block the cannula lumen 118, which may otherwise prevent, or at least make difficult, deployment of the tissue ablation probe 104.

The ablation probe 104 comprises an elongated shaft 126 having a distal end 128 and a proximal end 130. The diameter of the probe shaft 126 is sized to fit through the lumen 118 of the cannula 102, while the length of the probe shaft 126 is sized, such that its distal end 128 extends out from the exit port 120 of the cannula 102 when the probe 104 is fully introduced into the cannula 102. The probe shaft 126 is composed of a suitable plastic material, such as polyurethane, nylon, Pebax®, Hytrel®, etc. The distal end 128 of the probe shaft 126 is preferably stiff enough, so that it can be guided through the nucleus pulposus of the intervertebral disc without collapsing. For example, the distal end 128 of the probe shaft 126, or the entire length of the probe shaft 126, can include a braid (not shown) composed of a suitable material, such as Nylon or Kevlar, to increased its rigidity. The probe shaft 126 need not be capable of penetrating the annulus fibrosus of the intervertebral disc, since access to the nucleus pulposus will be provided via the cannula 102.

The ablation probe 104 further comprises a RF ablation electrode 132 mounted on the distal end 128 of the probe shaft 126. In the illustrated embodiment, the ablation electrode 132 takes the form of a hollow tip metal conducting cup mounted on the distal extremity of the probe shaft 126. In particular, as illustrated in FIG. 11, the tip electrode 132 comprises an outer wall 134 and a cavity 136 formed within the wall 134. The outer wall 134 can be formed of any suitable material, such as stainless steel, and has a suitable wall thickness, e.g., ranging from 0.003 to 0.004 inches. The outer wall 134 has a generally hemispherical configuration with a rounded exposed exterior surface 138 and a continuous cylindrical exposed exterior surface 140. The tip electrode 132 can be secured to the probe shaft 126 by any suitable means, such as bonding. As shown, the ablation probe 104 further comprises additional RF ablation electrodes 142, which are formed as spaced-apart bands provided on the exterior of the distal end 128 of the probe shaft 126 and in relatively close proximity to the tip electrode 132.

The probe 104 further comprises means for delivering RF ablation energy to the ablation electrodes 132, 142. In particular, as illustrated in FIG. 12, the probe 104 comprises a central lumen 144 that extends through the probe shaft 126, and a RF wire 146 that extends through the central lumen 144 in electrical contact with the tip electrode 132. Besides providing a means for delivering RF energy to the tip electrode 132, the RF wire 132 also serves to anchor the tip electrode 132, so that it remains secured to the distal extremity of the probe shaft 126. The probe 104 also comprises additional lumens 148 that extend through the probe shaft 126, and RF wires 150 that extend through the lumens 148 in electrical contact with the ring electrodes 142.

The probe 104 further comprises means for cooling the tip electrode 132 during the ablation process. In particular, the probe 104 comprises a pair of cooling and return lumens 152, 154 that extend through the probe shaft 126 in fluid communication with the cavity 136 of the tip electrode 132. As will be described in further detail below, a cooling medium can be conveyed through the cooling lumen 152 into the cavity 136 of the tip electrode 132. Heat generated in the tip electrode 132 during the ablation process is then absorbed into the cooling medium within the cavity 136, which is then conveyed out of the cavity 136, through the return lumen 154. As can be seen in FIG. 12, the cooling and return lumens 152, 154 are crescent-shaped lumens that surround the central lumen 144 in a side-by-side relationship. Alternatively, as illustrated in FIG. 13, cooling and return lumens 160, 162 can be arranged in a concentric relationship. In this case, the cooling medium is conveyed through the cooling lumen 160, into the cavity 136 of the tip electrode 132, and then out through the return lumen 162.

The probe 104 further comprises means for steering the distal end 128 of the probe shaft 126. In particular, the probe 104 comprises a pair of steering wire lumens 156 and a pair of steering wires 158 that extend through the lumens 156, terminating in plugs (not shown) suitably bonded at the distal ends of the lumens 156.

Referring back to FIG. 10, the probe 104 further comprises a handle 164 mounted to the proximal end 130 of the probe shaft 126. The handle 164 is preferably composed of a durable and rigid material, such as medical grade plastic, and is ergonomically molded to allow a physician to more easily manipulate the probe 104. Besides providing a means for grasping the probe 104, the handle 164 provides an interface between the probe 104 and the RF generator 106 and pump assembly 108. In particular, the handle 164 comprises an RF connector 166 in which the RF wires 146, 150 proximally terminate. The RF connector 166 is configured to mate with the RF generator 106 via an RF cable 168. The handle 164 further comprises tubular members 170, 172 that are in respective fluid communication with the cooling and return lumens 152, 154. The tubular members 170, 172 comprise luer connectors 174, 176 that are configured to mate with the pump assembly 108, as will be described in further detail below. The handle 164 also comprises a steering mechanism 178 in which the steering wires 158 proximally terminate. The steering mechanism 178 is configured to alternately pull the steering wires 158 in order to bend the distal end 128 of the probe shaft 126 in opposite directions, as illustrated in phantom in FIG. 10. Further details describing the structure and function of the steering mechanism 178 are disclosed in U.S. Pat. No. 5,871,525, which is expressly incorporated herein by reference.

The RF generator 106 may be a conventional RF power supply that operates at a frequency in the range from 200 KHz to 1.25 MHz, with a conventional sinusoidal or non-sinusoidal wave form. Such power supplies are available from many commercial suppliers, such as Valleylab, Aspen, and Bovie. Most general purpose electrosurgical power supplies, however, operate at higher voltages and powers than would normally be necessary or suitable for vessel occlusion. Thus, such power supplies would usually be operated at the lower ends of their voltage and power capabilities. More suitable power supplies will be capable of supplying an ablation current at a relatively low voltage, typically below 150V (peak-to-peak), usually being from 50V to 100V. The power will usually be from 20 W to 200 W, usually having a sine wave form, although other wave forms would also be acceptable. Power supplies capable of operating within these ranges are available from commercial vendors, such as Boston Scientific Corporation of San Jose, Calif., who markets these power supplies under the trademarks RF2000 (100 W) and RF3000 (200 W).

The pump assembly 108 comprises means for introducing a cooling medium into the cooling lumen 152 of the probe 104 via the handle 164. In particular, the pump assembly 108 comprises a tank 180, which contains a cooling medium 182, e.g., cooled saline solution, and a cooling pump 184. In the illustrated embodiment, the cooling medium 182 is cooled to a temperature ranging from 5° C. to 10° C. It should be appreciated liquids other than a saline solution can be utilized if desired. Also, the cooling medium 182 with the tank 180 may be maintained at other temperatures, e.g., room temperature. The cooling pump 184 comprises an inlet tubular member 186 in fluid communication with the tank 180 and an outlet tubular member 188 that is connected to the luer connector 174 of the tubular member 170 extending from the handle 164. Thus, it can be appreciated that operation of the cooling pump 184 conveys the cooling medium 182 from the tank 180, through the cooling lumen 152 within the probe shaft 126, and into cavity 136 of the tip electrode 132. In the illustrated embodiment, the cooling pump 184 delivers the cooling medium 182 to the probe 104 at a predetermined pressure, as measured by the pressure gauge 190.

In order to reduce the pressure of the cooling medium 182 in the probe 104, the pump assembly 108 further comprises means for withdrawing the cooling medium 182 from the return lumen 154 of the probe 104 via the handle 164. In particular, the pump assembly 108 comprises a return pump 192 with an outlet tubular member 194 in fluid communication with the tank 180 and an inlet tubular member 196 that is connected to the luer connector 176 of the tubular member 172 extending from the handle 164. Thus, it can be appreciated that operation of the return pump 192 conveys the cooling medium 182 (along with the heat absorbed from the tip electrode 132) from the cavity 136 of the tip electrode 132, through the return lumen 154, and back into the tank 180.

Further details on the construction of the tissue ablation system 100, as well as alternative embodiments, are disclosed in U.S. Pat. No. 5,697,927, which is fully and expressly incorporated herein by reference.

Having described the structure of the tissue ablation system 100, its operation will now be described with reference to FIGS. 14A-14E in treating a herniated intervertebral disc. First, the cannula 102 is introduced into the herniated disc 12′ via a extrapedicular posterior-lateral approach (FIG. 14A). Alternatively, a stylet (not shown) can be introduced through the cannula lumen (not shown in FIG. 14A) to facilitate introduction into the herniated disc 12′. As illustrated in FIG. 14A, the cannula 102 pierces the annulus fibrosus 42 and is advanced therethrough into the nucleus pulposus 40. Once the distal end 114 of the cannula 102 is advanced a desired distance into the nucleus pulposus 40, the ablation probe 104 is introduced through the cannula 102 until its distal end 128, and thus, the tip electrode 132, exits the distal end 114 of the cannula 102 into the nucleus pulposus 42 (FIG. 14B). In the illustrated method, the distal end 128 of the ablation probe 104 is placed in the center of the nucleus pulposus 40 away from the herniated region 46 of the disc 12′.

Next, the tissue ablation probe 104 is mated to the RF generator 106 and the pump assembly 108, which are then operated to ablate tissue within the center of the nucleus pulposus 40, thereby decompressing the disc 12′ (FIG. 14C). In particular, the RF generator 106 is operated to convey RF ablation energy to the tip electrode 132, and optionally, the ring electrodes 142, while the pump assembly 108 is operated to convey the cooling medium 182 in an out of the cavity 136 within the tip electrode 132, thereby cooling the tip electrode 132 during the ablation process. Thus, tissue charring is prevented, or at least minimized, thereby allowing RF energy to be conveyed from the tip electrode 132 to tissue regions not directly adjacent the tip electrode 132. As a result, a greater tissue volume of the nucleus pulposus 40 can be removed to decompress the disc 12′ without having to move the probe 104. In an alternative method, the ablation probe 104 is steered into contact with the herniated region 46 of the disc 12′ by operating the steering mechanism 178 on the handle 164 to bend the distal end 128 of the probe 104 while the probe 104 is advanced from the cannula 102 (FIG. 14D). Once the tip electrode 132 is positioned within the herniated region 46, the RF generator 106 and pump assembly 108 are operated to ablate the nucleus pulposus 40 within the herniated region 46 (FIG. 14E). Optionally, the RF generator 106 can be provided with an autosafety switch or mechanism that senses an uncontrolled burn that ablates neural tissue and shuts off the power accordingly.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. In addition, an illustrated embodiment needs not have all the aspects or advantages of the invention shown. An aspect or an advantage described in conjunction with a particular embodiment of the present invention is not necessarily limited to that embodiment and can be practiced in any other embodiments of the present invention even if not so illustrated. Thus, the present invention is intended to cover alternatives, modifications, and equivalents that may fall within the spirit and scope of the present invention as defined by the claims.

1. A method of treating an intervertebral disc, comprising:
introducing a probe with an electrode into contact with the nucleus pulposus of the disc;
conveying radio frequency ablation energy from the electrode into the nucleus pulposus; and
circulating a cooling medium through the probe in thermal contact with the electrode.

2. The method of claim 1, wherein the wherein the cooling medium is within the temperature range of 5° C.-10° C.

3. The method of claim 1, wherein the disc has a herniated region, the electrode is placed into contact with nucleus pulposus within the herniated region, and the ablation energy is conveyed directly into the herniated region.

4. The method of claim 3, further comprising steering the electrode into contact with the nucleus pulposus within the herniated region.

5. The method of claim 1, wherein the electrode is placed into contact with the nucleus pulposus near the center region of the disc, and the ablation energy is conveyed directly into the center region.

6. The method of claim 1, wherein the disc decompresses.

7. The method of claim 1, wherein the cooling fluid is circulated through the probe while the ablation energy is conveyed from the electrode.

8. The method of claim 1, wherein the probe is percutaneously introduced into the disc.

9. A method of treating an intervertebral disc having a herniated region, comprising:
introducing a probe into contact with the disc, the probe having a steerable distal tip and an ablative element located on the distal tip;
steering the distal tip of the probe into contact with the nucleus pulposus in the herniated region; and
conveying ablation energy from the ablative element directly into the herniated region.

10. The method of claim 9, further comprising circulating a cooling medium through the probe in thermal contact with the ablative element.

11. The method of claim 9, wherein the ablation energy is radio frequency ablation energy.

12. The method of claim 9, wherein the probe is percutaneously introduced into the disc.

13. The method of claim 9, wherein the herniated region is contained.

14. The method of claim 9, wherein the herniated region is not contained.

15. The method of claim 9, wherein the probe has a steering mechanism that is operated to steer the distal tip.
